(19) United States
(12) Patent Application Publication
(10) Pub. No.: US 2026/0137418 A1
(43) Pub. Date: May 21, 2026

(54) ATHERECTOMY BURR AXIALLY ALIGNED WITH DRIVESHAFT

(71) Applicant: Boston Scientific Scimed, Inc., Maple Grove, MN (US)

(72) Inventors: Daniel Frank Massimini, Brooklyn Park, MN (US); Gary Thomas Ohrt, Corcoran, MN (US); Michael Kaland, White Bear Lake, MN (US)

(73) Assignee: Boston Scientific Scimed, Inc., Maple Grove, MN (US)

(21) Appl. No.: 19/395,637

(22) Filed: Nov. 20, 2025

Related U.S. Application Data
(60) Provisional application No. 63/722,853, filed on Nov. 20, 2024.

(51) Int. Cl. A61B 17/3207; A61B 17/00; A61B 17/32

(52) U.S. Cl. CPC A61B 17/320758; A61B 2017/00398; A61B 2017/00477; A61B 2017/320052

(57) ABSTRACT

An atherectomy system includes an atherectomy burr and a driveshaft adapted to carry the atherectomy burr. The atherectomy burr includes a cavity that extends distally into the atherectomy burr from a proximal end region. The driveshaft extends into the cavity. An adhesive is within the cavity to secure the atherectomy burr to the driveshaft. At least one of the cavity and/or the driveshaft are adapted to align a centerline of the atherectomy burr with a centerline of the driveshaft.

### CROSS-REFERENCE TO RELATED APPLICATIONS

This application claims the benefit of priority under 35 U.S.C. §119 of U.S. Provisional Application No. 63/722,853, filed November 20, 2024, the entire disclosure of which is hereby incorporated by reference.

### TECHNICAL FIELD

The present disclosure pertains to medical devices, and methods for manufacturing and using medical devices. More particularly, the present disclosure pertains to rotational medical devices, methods, and systems.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an atherectomy system. The atherectomy system includes an atherectomy burr that extends from a proximal end region to a distal end region and that includes a burr centerline. A cavity extends distally into the atherectomy burr from the proximal end region and a driveshaft including a driveshaft centerline extends into the cavity. An adhesive is disposed within the cavity and secures the atherectomy burr to the driveshaft. At least one of the cavity and/or the driveshaft are adapted to align the driveshaft centerline with the burr centerline.

Alternatively or additionally, the atherectomy system may further include a guidewire lumen that extends distally from and coaxial with the cavity.

Alternatively or additionally, the atherectomy system may further include a drive assembly that is adapted to rotate the driveshaft over a guidewire extending through the guidewire lumen.

Alternatively or additionally, the cavity may include a proximal region having a first inner diameter and a distal region having a second inner diameter that is less than the first inner diameter.

Alternatively or additionally, the driveshaft may have an outer diameter that is equal to the second inner diameter.

Alternatively or additionally, the distal region may be created when removing material from the atherectomy burr to form the cavity.

Alternatively or additionally, the distal section may be formed by removing material from the atherectomy burr to form a constant diameter cavity, followed by disposing an annular ring within the cavity.

Alternatively or additionally, the cavity may have a constant inner diameter, the atherectomy burr may include an extension that extends proximally within the cavity and that defines an inner diameter that is aligned with the guidewire lumen as well as an outer diameter, and the driveshaft may include a proximal portion having an outer diameter equal to the constant inner diameter of the cavity, and a distal portion having a reduced inner diameter that matches the reduced inner diameter defined by the extension.

Alternatively or additionally, the distal portion of the driveshaft may be formed by removing material from an inner surface of the driveshaft.

Alternatively or additionally, the cavity may have a constant inner diameter. The driveshaft may include a proximal section having a constant outer diameter and a flared distal section that has an increasing outer diameter. The flared distal section may have a maximum outer diameter that is equal to the constant inner diameter of the cavity.

Alternatively or additionally, the cavity may have a constant inner diameter and the driveshaft may have a constant outer diameter. A metal ring having an outer diameter that is equal to the constant inner diameter of the cavity may be welded to the driveshaft.

Alternatively or additionally, the cavity may have a tapering inner diameter, with a maximum inner diameter at a proximal end of the cavity and a minimum inner diameter at a distal end of the cavity.

Alternatively or additionally, the cavity may include a proximal region having a first inner diameter, a distal region having a second inner diameter that is less than the first inner diameter, and an intermediate tapered region. The driveshaft may have an outer diameter that is equal to the second inner diameter.

Alternatively or additionally, the cavity may include a plurality of step-wise reductions in diameter, ranging from a maximum inner diameter at the proximal end of the cavity to a minimum inner diameter at the distal end of the cavity. The driveshaft may have an outer diameter equal to the minimum inner diameter.

Alternatively or additionally, the atherectomy burr may be symmetric about the burr centerline.

Alternatively or additionally, the atherectomy burr may be asymmetric about the burr centerline.

Another example may be found in an atherectomy system. The atherectomy system includes an atherectomy burr and a cavity that extends distally into the atherectomy burr. The cavity has a varying inner diameter including a minimum inner diameter. A driveshaft extends into the cavity and has an outer diameter equal to the minimum inner diameter. An adhesive is disposed within the proximal region of the cavity and secures the atherectomy burr to the driveshaft.

Alternatively or additionally, the driveshaft may include a drive coil.

Alternatively or additionally, the cavity may include a step-wise change in diameter from the proximal region to the distal region.

Another example may be found in an atherectomy system. The atherectomy system includes an atherectomy burr and a cavity that extends distally into the atherectomy burr. The cavity includes a proximal region having a proximal region inner diameter and a distal region having a distal region inner diameter that is less than the proximal region inner diameter. A driveshaft having an outer diameter equal to the distal region inner diameter extends into the cavity. An adhesive secures the atherectomy burr to the driveshaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic diagram showing an illustrative atherectomy system;

FIG. 2 is a schematic diagram showing features of the illustrative atherectomy system of FIG. 1;

FIG. 3 is a schematic cross-sectional view showing an illustrative atherectomy system;

FIG. 4 is a schematic cross-sectional view showing an illustrative atherectomy system;

FIG. 5 is a schematic cross-sectional view showing an illustrative atherectomy system;

FIG. 6 is a schematic cross-sectional view showing an illustrative atherectomy system;

FIG. 7 is a schematic cross-sectional view showing an illustrative atherectomy system;

FIG. 8 is a schematic cross-sectional view showing an illustrative atherectomy system;

FIG. 9 is a schematic cross-sectional view showing an illustrative atherectomy system;

FIG. 10 is a schematic cross-sectional view showing an illustrative atherectomy system; and

FIG. 11 is a schematic cross-sectional view showing an illustrative atherectomy system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Cardiovascular disease and peripheral arterial disease may arise from accumulation of atheromatous material on the inner walls of vascular lumens, resulting in a condition known as atherosclerosis. Atheromatous and other vascular deposits may restrict blood flow and can cause ischemia in a heart of a patient, vasculature of a patient’s legs, a patient’s carotid artery, etc. Such ischemia may lead to pain, swelling, wounds that will not heal, amputation, stroke, myocardial infarction, and/or other conditions.

Atheromatous deposits may have widely varying properties, with some deposits being relatively soft and others being fibrous and/or calcified. In the latter case, the deposits may be referred to as plaque. Atherosclerosis occurs naturally because of aging, but may also be aggravated by factors such as diet, hypertension, heredity, vascular injury, and the like. Atherosclerosis may be treated in a variety of ways, including drugs, bypass surgery, and/or a variety of catheter-based approaches that may rely on intravascular widening or removal of the atheromatous or other material occluding the blood vessel. Atherectomy is a catheter-based intervention that may be used to treat atherosclerosis.

Atherectomy is an interventional medical procedure performed to restore a flow of blood through a portion of a patient’s vasculature that has been blocked by plaque or other material (e.g., blocked by an occlusion). In an atherectomy procedure, a device on an end of a driveshaft is used to engage and/or remove (e.g., abrade, grind, cut, shave, etc.) plaque or other material from a patient’s vessel (e.g., artery or vein). In some cases, the device on an end of the driveshaft may be abrasive and/or may otherwise be configured to remove plaque from a vessel wall or other obstruction in a vessel when the device is rotating and engages the plaque or other obstruction. In some cases, atherectomy involves using an abrasive atherectomy burr that is rotated at high speeds exceeding 100,000 revolutions per minute (RPM) in order to abrade plaque and other hardened materials from within the patient’s vessel. Atherectomy burrs may be rotated at speeds exceeding 140,000 RPM, at speeds exceeding 180,000 RPM and even at speeds as high as 220,000 RPM. Atherectomy may include orbital atherectomy in addition to rotational atherectomy.

In some instances, an atherectomy system may include an atherectomy burr that extends from a proximal end region to a distal end region and that includes a burr centerline. A cavity extends distally into the atherectomy burr from the proximal end region. A driveshaft extends into the cavity, with the driveshaft including a driveshaft centerline. An adhesive is disposed within the cavity and secures the atherectomy burr to the driveshaft. At least one of the cavity and/or the driveshaft are adapted to align the driveshaft centerline with the burr centerline. In some cases, the atherectomy system may further include a guidewire lumen that extends distally from and coaxial with the cavity. In some cases, the atherectomy system may further include a drive assembly that is adapted to rotate the driveshaft over a guidewire extending through the guidewire lumen. In some cases, the atherectomy burr may be symmetric about the burr centerline. In some cases, the atherectomy burr may be asymmetric about the burr centerline.

In some cases, the cavity may include a proximal region having a first inner diameter and a distal region having a second inner diameter that is less than the first inner diameter. The driveshaft may include a proximal section having a first outer diameter equal to the first inner diameter of the proximal region and a distal section having a second outer diameter equal to the second inner diameter. In some cases, the distal region may be created when removing material from the atherectomy burr to form the cavity. In some cases, the distal section may be formed by removing material from the atherectomy burr to form a constant diameter cavity, followed by disposing an annular ring within the cavity.

In some cases, the cavity may have a constant inner diameter. The atherectomy burr may include an extension that extends proximally within the cavity and that defines an inner diameter that is aligned with the guidewire lumen and an outer diameter. The driveshaft may include a proximal portion having an outer diameter equal to the constant inner diameter of the cavity, and a distal portion having a reduced inner diameter that matches the reduced inner diameter defined by the extension. In some cases, the distal portion of the driveshaft may be formed by removing material from an inner surface of the driveshaft.

In some cases, the cavity may have a constant inner diameter and the driveshaft may include a proximal section having a constant outer diameter and a flared distal section having an increasing outer diameter. The flared distal section may have a maximum outer diameter that is equal to the constant inner diameter of the cavity. In some cases, the cavity may have a constant inner diameter and the driveshaft may have a constant outer diameter. A metal ring may be welded to the driveshaft. The metal ring may have an outer diameter that is equal to the constant inner diameter of the cavity.

In some cases, the cavity may have a tapering inner diameter, with a maximum inner diameter at a proximal end of the cavity and a minimum inner diameter at a distal end of the cavity. In some cases, the cavity may include a proximal region having a first inner diameter, a distal region having a second inner diameter that is less than the first inner diameter, and an intermediate tapered region. The driveshaft may have an outer diameter that is equal to the second inner diameter. In some cases, the cavity may include a plurality of step-wise reductions in diameter, ranging from a maximum inner diameter at the proximal end of the cavity to a minimum inner diameter at the distal end of the cavity. The driveshaft may have an outer diameter that is equal to the minimum inner diameter.

In some instances, an atherectomy system includes an atherectomy burr and a cavity extending distally into the atherectomy burr. The cavity has a varying inner diameter including a minimum inner diameter. A driveshaft extends into the cavity, the driveshaft having an outer diameter equal to the minimum inner diameter. An adhesive is disposed within the proximal region of the cavity and secures the atherectomy burr to the driveshaft. In some cases, the driveshaft may include a drive coil. In some cases, the cavity may include a step-wise change in diameter from the proximal region to the distal region.

In some instances, an atherectomy system includes an atherectomy burr and a cavity that extends distally into the atherectomy burr. The cavity includes a proximal region having a proximal region inner diameter and a distal region having a distal region inner diameter that is less than the proximal region inner diameter. A driveshaft extends into the cavity. The driveshaft has an outer diameter that is equal to the distal region inner diameter of the distal region of the cavity. An adhesive is within the cavity, securing the atherectomy burr to the driveshaft.

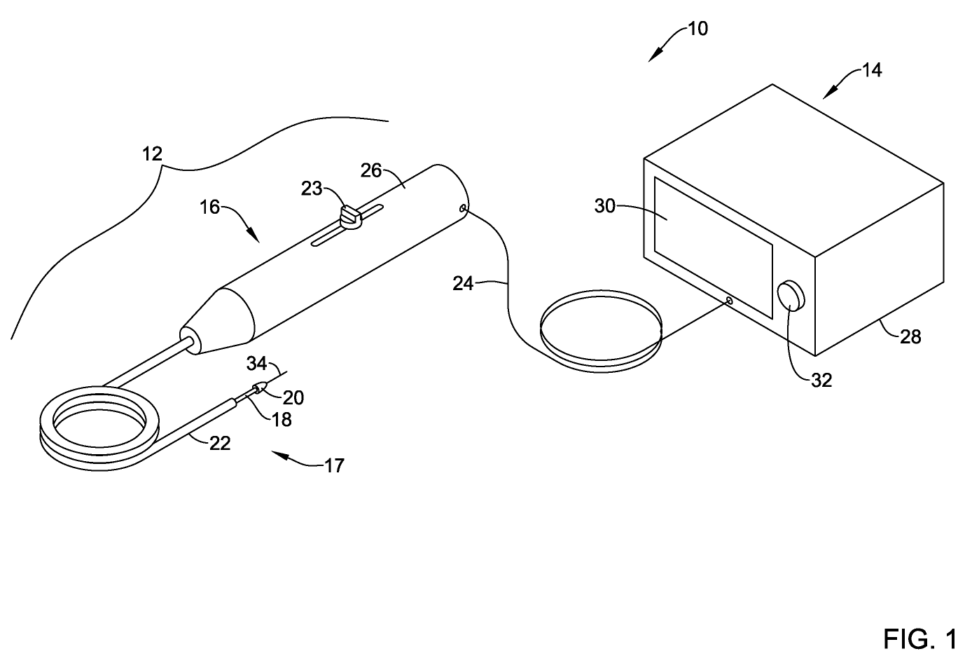

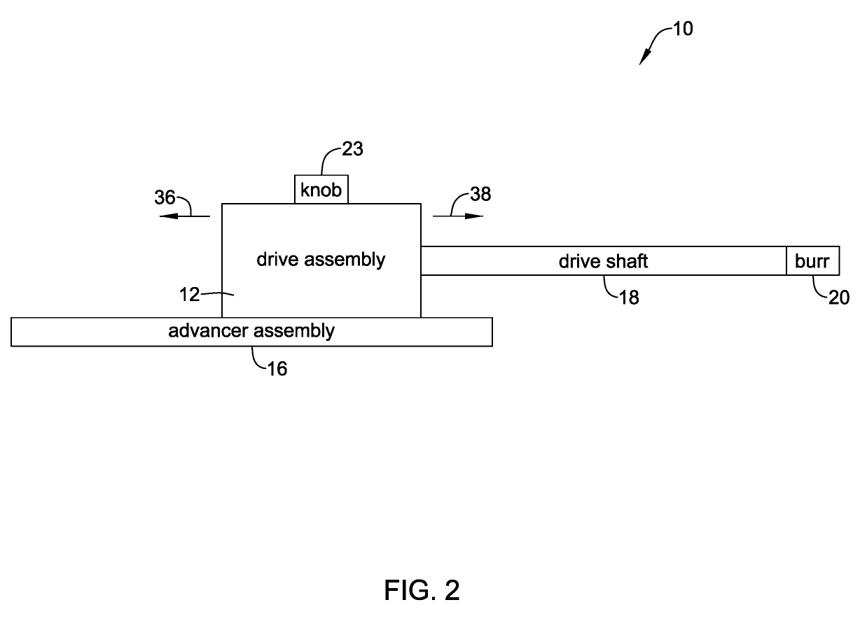

FIGS. 1 and 2 depict an atherectomy system 10. The atherectomy system 10 may be electrically driven, pneumatically driven and/or driven in one or more other suitable manners. Additional or alternative components to those illustrated and described herein may be utilized in the operation of the atherectomy system 10. The atherectomy system 10 may include a drive assembly 12 and a control unit 14 (e.g., a controller). The drive assembly 12 may include, among other elements, an advancer assembly 16 and a rotation assembly 17. Although the control unit 14 is depicted as being separate from the drive assembly 12 in FIG. 1, the functionality of the control unit 14 and the drive assembly 12 may be incorporated into a single component (e.g., in the advancer assembly 16 or other suitable single component).

The rotation assembly 17 may include a driveshaft 18 (e.g., an elongate member that may be or may include a flexible driveshaft or other suitable driveshaft), an atherectomy burr 20 and an elongate member 22 having a first end (e.g., a proximal end), a second end (e.g., a distal end), and a lumen extending from the first end to the second end for receiving the driveshaft 18. In some cases, the elongate member 22 may be an elongated tubular member. The driveshaft 18 may include a lumen extending therethrough (not shown) in order to accommodate a guidewire 34. In some cases, the guidewire 34 may be advanced through the vasculature to reach a desired treatment site, and then the driveshaft 18 (with the atherectomy burr 20 secured thereto) may be advanced over the guidewire 34. In some cases, the driveshaft 18 and the atherectomy burr 20 may be rotated over the guidewire 34 during use. The atherectomy burr 20 may have a rough or sharp surface, such that it is configured to grind, abrade, cut, shave, etc. plaque from a vessel wall or other obstruction in a vessel when it is rotated.

In some instances, the driveshaft 18 may be a coil spring such as a drive coil. As such, it will be appreciated that rotation of the driveshaft 18 in a first direction may result in the driveshaft 18 undergoing compression, particularly when the first direction corresponds to tightening the individual windings of the coil spring. Rotation of the driveshaft 18 in a second, opposing, direction may result in the driveshaft 18 undergoing tension, particularly when the second direction corresponds to loosening the individual windings of the coil spring.

The advancer assembly 16 may include a knob 23, a housing 26, the drive assembly 12 and/or one or more other suitable components. In some instances, the drive assembly 12 may be or may include a motor (e.g., an electric motor, pneumatic motor, or other suitable motor) at least partially housed within the housing 26 and in communication with the knob 23, the driveshaft 18, and the control unit 14. In some cases, the motive force may not be disposed within the drive assembly 12, but may instead be remotely located, with a flexible drive cable extending from the motive force to the drive assembly 12. The knob 23 may be configured to advance along a longitudinal path to longitudinally advance the drive assembly 12 and the rotation assembly 17. The housing 26 may at least partially house the drive assembly 12 and the knob 23 may be at least partially accessible from an exterior of the housing 26.

In some instances, the drive assembly 12 is adapted to be translationally secured relative to an advancer assembly 16. In some instances, the advancer assembly 16 may be adapted to be fixed in space, such as being secured to a table, for example. In some instances, the advancer assembly 16 may be part of an advancer housing such as the housing 26. The drive assembly 12 may also be disposed within an advancer handle, for example, but is adapted to translate back and forth (left and right in the illustrated orientation) as indicated by arrows 36 and 38 in response to a user moving the knob 23 in the directions indicated by the arrows 36 and 38. In some instances, as the drive assembly 12 moves back and forth, the driveshaft 18 also moves correspondingly.

The drive assembly 12 may be coupled to the driveshaft 18 in a suitable manner including, but not limited to, a weld connection, a clamping connection, an adhesive connection, a threaded connection, and/or other suitable connection configured to withstand rotational speeds and forces. The driveshaft 18 may be formed from one or more of a variety of materials. For example, the driveshaft 18 may be formed from one or more of a variety of materials, including steel, stainless steel, other metal, polymer, and/or other suitable materials. The driveshaft 18 may have a suitable diameter and/or length for traversing vasculature of a patient. The diameter and/or the length of the driveshaft 18 may depend on the dimension of the lumen of the elongate member 22, the dimensions of vessels of a patient to be traversed, and/or one or more other suitable factors. In some cases, the driveshaft 18 may have a diameter in a range from about 0.0118 inches (0.030 centimeters (cm)) or smaller to about 0.0591 inches (0.150 cm) or larger and a working length in a range from about 3.937 inches (10) cm or shorter to about 118.1102 inches (300 cm) or longer. In one example, the driveshaft 18 may have a diameter of about 0.0255 inches (0.06477 cm). Alternatively, the driveshaft 18 may have a different suitable diameter and/or different suitable length.

The atherectomy burr 20 may have an outer perimeter which is equal to or larger than a distal diameter of the driveshaft 18 and/or the elongate member 22. Alternatively or in addition, the atherectomy burr 20 may have an outer perimeter which is smaller than a diameter of the driveshaft 18 and/or the elongate member 22. The atherectomy burr 20 may be coupled to the driveshaft 18. Where the driveshaft 18 has a first end portion (e.g., a proximal end portion) and a second end portion (e.g., a distal end portion), the atherectomy burr 20 may be coupled to the driveshaft 18 at or near the second end portion. In some cases, the atherectomy burr 20 may be located at or adjacent a terminal end of the second end portion of the driveshaft 18.

The drive assembly 12 and the control unit 14 may be in communication and may be located in or may have a same housing and/or located in or have separate housings (e.g., the advancer assembly housing 26 and a control unit housing 28 or other housings). Whether in the same housing or in separate housings, the drive assembly 12 and the control unit 14 may be in communication through a wired connection (e.g., via one or more wires in an electrical connector 24 or other suitable electrical connector) and/or a wireless connection. Wireless connections may be made via one or more communication protocols including, but not limited to, cellular communication, ZigBee, Bluetooth, Wi-Fi, Infrared Data Association (IrDA), dedicated short range communication (DSRC), EnOcean, and/or any other suitable common or proprietary wireless protocol, as desired.

Although not necessarily shown in FIG. 1, the drive assembly 12 may include and/or enclose one or more operational features. For example, among other features, the drive assembly 12 may include a motor (e.g., as discussed above and/or other suitable motor), rubber feet, control electronics, drive circuitry, etc.

The control unit 14, which may be separate from the drive assembly 12 (e.g., as shown in FIG. 1) or may be included in the drive assembly 12, may include several features. For example, as shown in FIG. 1, the control unit 14 may include a display 30 and a control knob 32 (e.g., a motor speed (e.g., RPM or other speed) adjustment knob or other control knob). Additionally or alternatively, the control unit 14 may include one or more other features for controlling the drive mechanism and/or other features of the drive assembly 12 (e.g., one or more drive mechanism states of the drive mechanism) including, but not limited to, a processor, memory, input/output devices, a speaker, volume control buttons, on/off power supply switch, motor activation switch, a timer, a clock, and/or one or more other features.

In some cases, the control unit 14 may include one or more drive load output control mechanisms for controlling an operation of the atherectomy system 10. In one example of a drive load output control mechanism that may be included in the control unit 14, the control unit 14 may include a mechanism configured to set and/or adjust an advancing load output (e.g., a rotational speed) and/or a retracting load output from the drive assembly 12. Additionally or alternatively, the control unit 14 may include other control and/or safety mechanism for controlling the operation of the atherectomy system 10 and mitigating risks to patients.

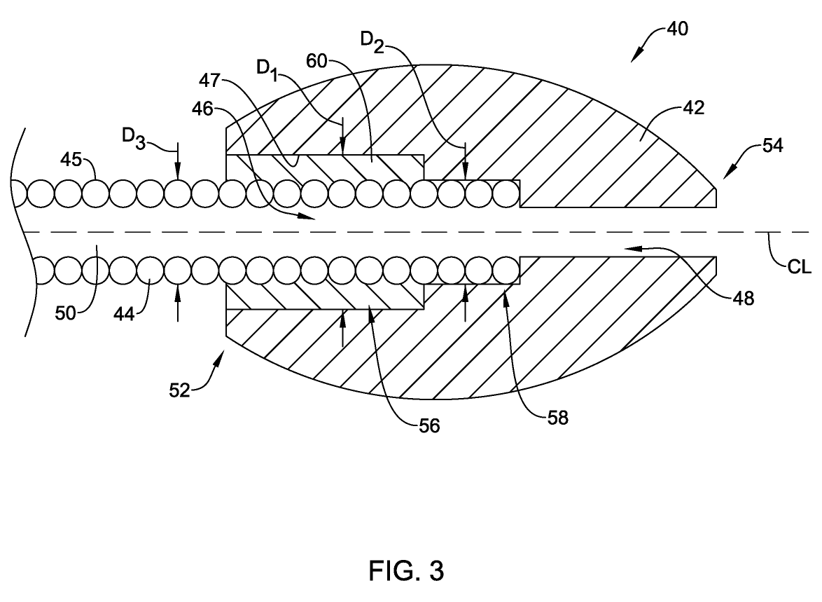

The atherectomy burr 20 may be coupled to the driveshaft 18 in any manner. For example, the atherectomy burr 20 may be coupled to the drive haft 18 with an adhesive connection, a threaded connection, a weld connection, a clamping connection, and/or other suitable connection configured to withstand rotational speeds and forces. Similar to as discussed above with respect to the connection between the driveshaft 18 and the drive mechanism, as the driveshaft 18 and/or the atherectomy burr 20 may rotate at speeds between zero (0) RPM and 250,000 RPM or higher, the coupling between the driveshaft 18 and the atherectomy burr 20 may be configured to withstand such rotational speeds and associated forces. In some cases, the atherectomy burr 20 may be adhesively secured to the driveshaft 18. Because of the high speeds at which the driveshaft 18 and the atherectomy burr 20 are rotated, there is a desire for the atherectomy burr 20 to be accurately positioned relative to the driveshaft 18. As will be discussed with respect to FIGS. 3 through 10, the atherectomy burr 20 includes a cavity formed within the atherectomy burr 20 that accommodates a portion of the driveshaft 18 that extends into the cavity.

When the atherectomy burr 20 is adhesively secured to the driveshaft 18, a tolerance exists between the outer diameter of the driveshaft 18 and an inner diameter of the cavity in order to provide space for an adhesive. Unfortunately, the space provided by this tolerance can mean that the atherectomy burr 20 may be secured in position on the driveshaft 18 in an orientation that does not align a centerline (or axis of rotation) of the atherectomy burr 20 with a centerline (or axis of rotation) of the driveshaft 18. The space allotted for adhesive may allow the atherectomy burr 20 to be offset, or moved radially from an alignment of the centerlines of the atherectomy burr 20 and the driveshaft 18. The space allotted for adhesive may allow the atherectomy burr 20 to be tilted, or angled with respect to the driveshaft 18. FIGS. 3 through 10 provide examples of the atherectomy burr 20 and/or the driveshaft 18 being adapted to cause a centerline (or axis of rotation) of the atherectomy burr 20 to be coaxially aligned with a centerline (or axis of rotation) of the driveshaft 18.

FIG. 3 is a schematic cross-sectional view of an illustrative atherectomy system 40. The illustrative atherectomy system 40 includes an atherectomy burr 42 that is secured to a driveshaft 44. The atherectomy system 40 may be used with the atherectomy system 10 shown in FIGS. 1 and 2, with the atherectomy burr 42 replacing the atherectomy burr 20 and the driveshaft 44 replacing the driveshaft 18. As shown, the driveshaft 44 is a drive coil. The atherectomy burr 42 defines a cavity 46 that extends distally into the atherectomy burr 42. A guidewire lumen 48 extends distally from the cavity 46 and is coaxially aligned with the cavity 46. The guidewire lumen 48 may be considered as being coaxially aligned with a lumen 50 that extends through the driveshaft 44. This allows the atherectomy system 40 to be advanced over the guidewire 34 (FIG. 1). The atherectomy burr 42 may be considered as extending from a proximal end region 52 to a distal end region 54. A centerline CL extends through the atherectomy system 40. Because the atherectomy burr 42 is appropriately aligned with the driveshaft 44, the centerline CL represents both a centerline (or axis of rotation) of the driveshaft 44 as well as a centerline (or axis of rotation) of the atherectomy burr 42.

As shown, the cavity 46 includes a proximal region 56 that has a first inner diameter D₁ and a distal region 58 that has a second inner diameter D₂. In some cases, the second inner diameter D₂ is less than the first inner diameter D₁. In some cases, the distal region 58 having the second inner diameter D₂ may be formed during a process in which the cavity 46, including the proximal region 56 and the distal region 58, are formed by removing material from the atherectomy burr 42. This may occur via drilling, grinding or milling, for example. In some cases, the distal region 58 may have a second inner diameter D₂ that is less than the first inner diameter D₁ as a result of an annular ring being disposed within the cavity 46 to form the distal region 58. In some cases, the driveshaft 44 has an outer diameter D₃. In some cases, the outer diameter D₃ is equal or substantially equal to the second inner diameter D₂ . Equal or substantially equal may be defined as the second inner diameter D₂ being within 0.0050 inches (0.13 millimeters) ± or less of the outer diameter D₃. For example, the second inner diameter D₂ may be up to 0.0050 inches (0.013 centimeters) smaller or larger than the outer diameter D₃.

In some cases, D₃ may vary from about 0.0118 inches (0.030 cm) or smaller to about 0.0591 inches (0.150 cm) or larger. D₂ may vary from D₃ minus 0.005 inches (0.013 cm) to D₃ plus 0.005 inches (0.013 cm). Accordingly, D₂ may range from 0.0068 inches (0.0172 cm) to 0.0641 inches (0.163 cm). D₁ may vary from D₃ to D₃ plus 0.015 inches (0.0381 cm). Accordingly, D₁ may vary from 0.0118 inches (0.030 cm) to about 0.0741 inches (0.188 cm).

In some cases, the driveshaft 44 will fit tightly into the distal region 58, thereby allowing the driveshaft 18 to be inserted into the distal region 58 while also aligning the atherectomy burr 42 with the driveshaft 44 such that a centerline (or axis of rotation) of the atherectomy burr 42 and a centerline (or axis of rotation) of the driveshaft 18 both align with the centerline CL of the atherectomy system 40. In some cases, the centerline (or axis of rotation) of the atherectomy burr 42 will align within about ten percent or less, or about nine percent or less, or about eight percent or less, or about seven percent or less, or about six percent or less, or about five percent or less, or four percent or less, of a centerline (or axis of rotation) of the driveshaft 18.

As can be seen, the first inner diameter D₁ is larger than the outer diameter D₃, defining a space between an outer surface 45 of the driveshaft 18 and an inner surface 47 of the proximal region 56 that allows for an adhesive 60 to be positioned within the cavity 46, between the atherectomy burr 42 and the driveshaft 44. The adhesive 60 secured the atherectomy burr 42 to the driveshaft 44. A variety of different adhesive materials may be used as the adhesive 60. Examples include Master Bond® EP17HT-LO, Master Bond® EP45HTAN, 3M® Scotch-Weld 2241 Hi Temp New Formula, Adhesive Systems Inc.® MP 5401-4, MasterSil® 800, MasterSil® 972TC-LO and others.

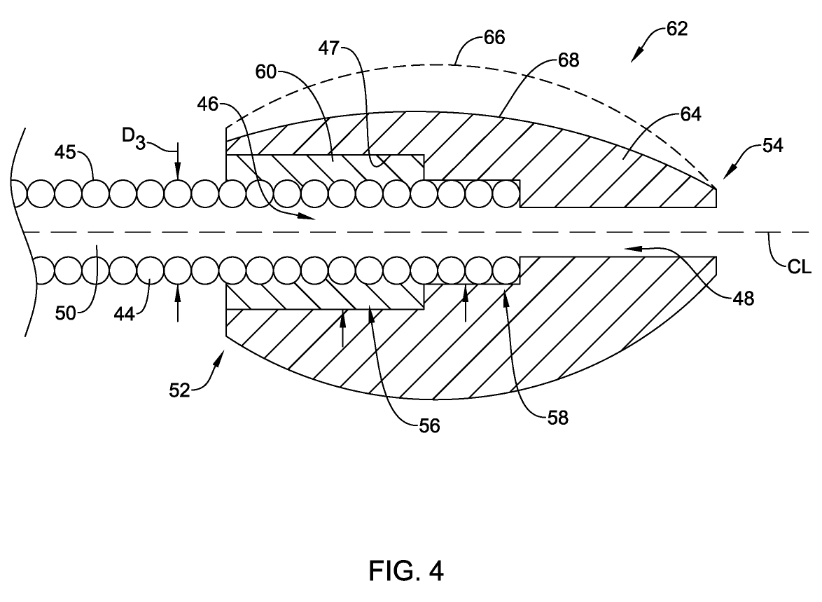

FIG. 4 is a schematic cross-sectional view of an illustrative atherectomy system 62. The illustrative atherectomy system 62 includes an atherectomy burr 64 that is secured to the driveshaft 44. The atherectomy system 62 may be used with the atherectomy system 10 shown in FIGS. 1 and 2, with the atherectomy burr 64 replacing the atherectomy burr 20 and the driveshaft 44 replacing the driveshaft 18. The atherectomy burr 64 includes the same cavity 46, including the proximal region 56 and the distal region 58. Accordingly, the atherectomy burr 64 is secured to the driveshaft 44 in the same manner as discussed with respect to the atherectomy burr 42 shown in FIG. 3. A centerline CL extends through the atherectomy system 62. Because the atherectomy burr 64 is appropriately aligned with the driveshaft 44, the centerline CL represents both a centerline (or axis of rotation) of the driveshaft 44 as well as a centerline (or axis of rotation) of the atherectomy burr 64.

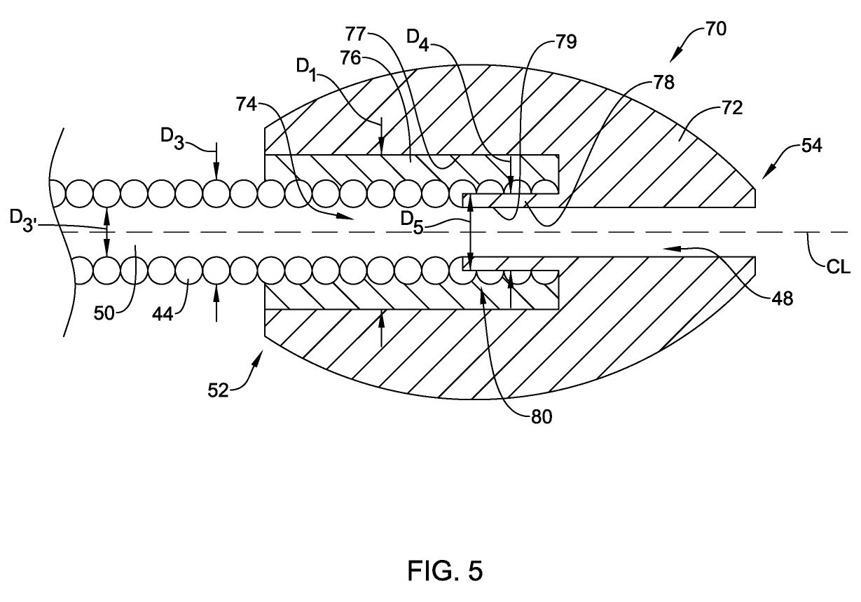

However, unlike the atherectomy burr 42, which may be considered as being symmetric about the centerline, the atherectomy burr 64 is not symmetric about the centerline. A dashed line 66 represents, relative to an outer surface 68, an amount of material that has been removed from the atherectomy burr 64. This means that upon rotation, the atherectomy burr 64 will not simply spin in place, but will oscillate in a circumferential manner while rotating. So, even though there is a desire for the atherectomy burr 64 to be aligned correctly with the driveshaft 44, it is still possible for the atherectomy burr 64 itself to be rotationally asymmetric. While the asymmetric atherectomy burr 64 is shown with respect to a particular securement between the atherectomy burr 64 and the driveshaft 44, it will be considered that any of the particular securements between the atherectomy burr and the driveshaft as will be shown in FIGS. 5 through 11 may also be used for securing an asymmetric atherectomy burr such as the atherectomy burr 64.

FIG. 5 is a schematic cross-sectional view of an illustrative atherectomy system 70. The illustrative atherectomy system 70 includes an atherectomy burr 72 that is secured to the driveshaft 44. The atherectomy system 70 may be used with the atherectomy system 10 shown in FIGS. 1 and 2, with the atherectomy burr 72 replacing the atherectomy burr 20 and the driveshaft 44 replacing the driveshaft 18. The atherectomy burr 72 defines a cavity 74 that extends distally into the atherectomy burr 72. The guidewire lumen 48 extends distally from the cavity 74 and is coaxially aligned with the cavity 74. The guidewire lumen 48 may be considered as being coaxially aligned with the lumen 50 that extends through the driveshaft 44. This allows the atherectomy system 70 to be advanced over the guidewire 34 (FIG. 1). The atherectomy burr 72 may be considered as extending from a proximal end region 52 to a distal end region 54. A centerline CL extends through the atherectomy system 70. Because the atherectomy burr 72 is appropriately aligned with the driveshaft 44, the centerline CL represents both a centerline (or axis of rotation) of the driveshaft 44 as well as a centerline (or axis of rotation) of the atherectomy burr 42.

As shown, the cavity 74 has an inner diameter D₁ that is greater than an outer diameter D₃ of the driveshaft 44. In some cases, the cavity 74 has a constant inner diameter D₁ . This allows an adhesive 76 to extend between an inner surface 75 of the cavity 74 and an outer surface 45 of the driveshaft 44. In order to accurately locate and position the atherectomy burr 72 relative to the driveshaft 44, the atherectomy burr 72 includes an extension 78 that extends proximally into the cavity 74. In some cases, the extension 78 may have a length of about 0.5 times the diameter D₃ to about 3 times the diameter D₃. The extension 78 defines an outer diameter D₄ in the atherectomy burr 72. The extension 78 also defines an inner diameter that aligns with the guidewire lumen 48. The driveshaft 44 includes a region 80 that is milled, ground, or otherwise processed to have an inner diameter D₅ that is equal or substantially equal to the outer diameter D₄. A tolerance between the outer diameter D₄ and the inner diameter D₅ allows the atherectomy burr 72 to be appropriately positioned relative to the drive shaft 44 such that a centerline (or axis of rotation) of the driveshaft 44 and a centerline (or axis of rotation) of the atherectomy burr 72 both align with the centerline CL of the atherectomy system 70. Equal or substantially equal may be defined as the outer diameter D₄ being within 0.0050 inches (0.13 millimeters) ± or less of the inner diameter D₅. For example, the outer diameter D₄ may be up to 0.0050 inches (0.13 millimeters) smaller or larger than the inner diameter D₅. In some cases, the driveshaft 44 will fit tightly, thereby allowing the driveshaft 18 to be inserted into while also aligning the atherectomy burr 72 with the driveshaft 44 such that a centerline (or axis of rotation) of the atherectomy burr 42 and a centerline (or axis of rotation) of the driveshaft 44 both align with the centerline CL of the atherectomy system 40.

The driveshaft 44 has an outer diameter D₃ and an inner diameter D₃’. In general, the extension 78 defines an outer surface 77 and an inner surface 79. The outer surface 77 defines how far the extension 78 extends into a milled out region of the driveshaft 44. The inner surface 79 generally aligns with the guidewire lumen 48. The diameter D₅ will vary from D₃’ to about half of the thickness of the driveshaft 44, defined as a difference between the diameter D₃ and the diameter D₃’. In some cases, the diameter D₄ and the diameter D are equal or substantially equal to each other, defined as the diameter D₄ being plus or minus within 0.0050 inches (0.013 cm) of the diameter D₅.

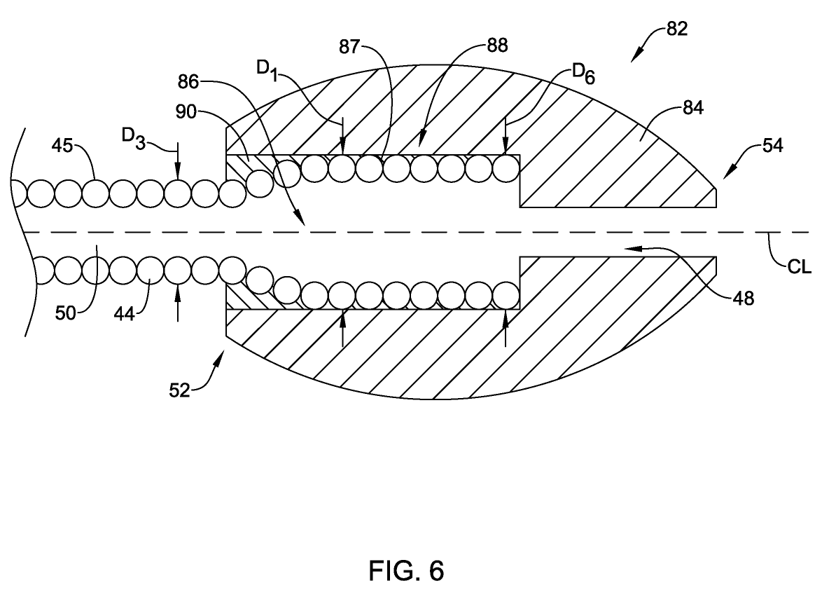

FIG. 6 is a schematic cross-sectional view of an illustrative atherectomy system 82. The illustrative atherectomy system 82 includes an atherectomy burr 84 that is secured to the driveshaft 44. The atherectomy system 82 may be used with the atherectomy system 10 shown in FIGS. 1 and 2, with the atherectomy burr 84 replacing the atherectomy burr 20 and the driveshaft 44 replacing the driveshaft 18. The atherectomy burr 84 defines a cavity 86 that extends distally into the atherectomy burr 84. The guidewire lumen 48 extends distally from the cavity 86 and is coaxially aligned with the cavity 86. The guidewire lumen 48 may be considered as being coaxially aligned with the lumen 50 that extends through the driveshaft 44. This allows the atherectomy system 82 to be advanced over the guidewire 34 (FIG. 1). The atherectomy burr 84 may be considered as extending from a proximal end region 52 to a distal end region 54. A centerline CL extends through the atherectomy system 82. Because the atherectomy burr 84 is appropriately aligned with the driveshaft 44, the centerline CL represents both a centerline (or axis of rotation) of the driveshaft 44 as well as a centerline (or axis of rotation) of the atherectomy burr 84.

As shown, the cavity 86 has an inner diameter D₁ that is constant. The driveshaft 44, however, includes a flared distal section 88 having an increasing outer diameter that terminates at a maximum outer diameter D₆. The maximum outer diameter D₆ may be equal or substantially equal to the inner diameter D_{1.} Equal or substantially equal may be defined as the maximum outer diameter D₆ being within 0.0050 inches (0.13 millimeters) ± or less of the inner diameter D₁. For example, the maximum outer diameter D₆may be up to 0.0050 inches (0.13 millimeters) smaller or larger than the inner diameter D₁. In some cases, the driveshaft 44 will fit tightly, thereby allowing the driveshaft 44 to be inserted into while also aligning the atherectomy burr 84 with the driveshaft 44 such that a centerline (or axis of rotation) of the atherectomy burr 84 and a centerline (or axis of rotation) of the driveshaft 44 both align with the centerline CL of the atherectomy system 82. An adhesive 90 is disposed between an inner surface 87 of the cavity 86 and an outer surface 45 of the driveshaft 44.

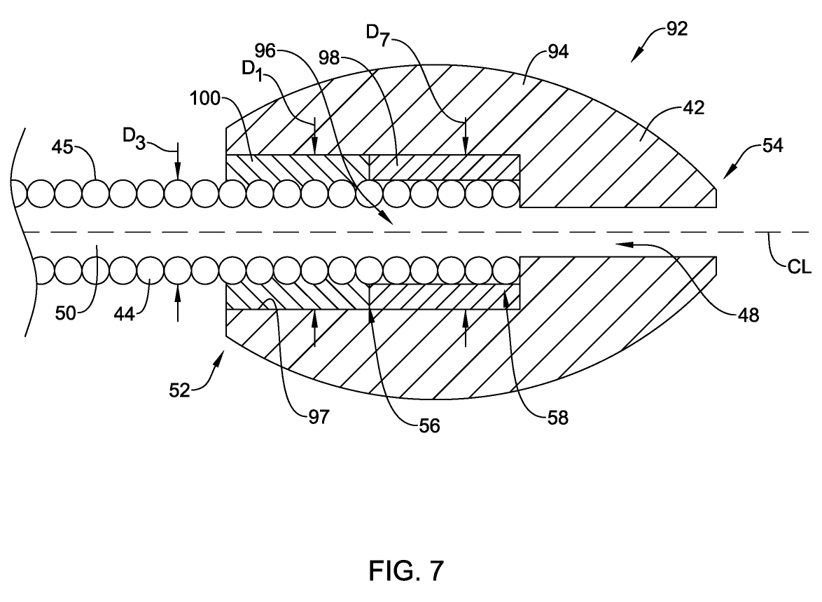

FIG. 7 is a schematic cross-sectional view of an illustrative atherectomy system 92. The illustrative atherectomy system 92 includes an atherectomy burr 94 that is secured to the driveshaft 44. The atherectomy system 92 may be used with the atherectomy system 10 shown in FIGS. 1 and 2, with the atherectomy burr 94 replacing the atherectomy burr 20 and the driveshaft 44 replacing the driveshaft 18. The atherectomy burr 94 defines a cavity 96 that extends distally into the atherectomy burr 94. The guidewire lumen 48 extends distally from the cavity 96 and is coaxially aligned with the cavity 96. The guidewire lumen 48 may be considered as being coaxially aligned with the lumen 50 that extends through the driveshaft 44. This allows the atherectomy system 92 to be advanced over the guidewire 34 (FIG. 1). The atherectomy burr 94 may be considered as extending from a proximal end region 52 to a distal end region 54. A centerline CL extends through the atherectomy system 92. Because the atherectomy burr 94 is appropriately aligned with the driveshaft 44, the centerline CL represents both a centerline (or axis of rotation) of the driveshaft 44 as well as a centerline (or axis of rotation) of the atherectomy burr 94.

The cavity 96 has a constant inner diameter D₁ that is greater than the outer diameter D₃ of the driveshaft 44. In some cases, an annular ring 98 may be welded to the driveshaft 44. The annular ring 98 may be formed of a metal that is the same as that used to form the driveshaft 44. As an example, the annular ring 98 may be formed of stainless steel such as SS 304 or SS 316. The annular ring 98 may be formed of nitinol, titanium or brass, for example. As shown, the annular ring 98 may be considered as having an outer diameter D₇that is equal to the inner diameter D₁ of the cavity 96. Equal or substantially equal may be defined as the constant inner diameter D₁ being within 0.0050 inches (0.13 millimeters) ± or less of the outer diameter D₃. For example, the constant inner diameter D₁ may be up to 0.0050 inches (0.13 millimeters) smaller or larger than the outer diameter D₃. Accordingly, an outer surface of the annular ring 98, which may for example be a metal ring, forms a tight fit within the cavity 96 in order to appropriately position the driveshaft 44 such that a centerline (or axis of rotation) of the atherectomy burr 94 aligns with the centerline (or axis of rotation) of the driveshaft 44, and thus align with the centerline CL of the atherectomy system 92. An adhesive 100 is disposed within the cavity 96, between an inner surface 97 of the cavity 96 and an outer surface 45 of the driveshaft 44.

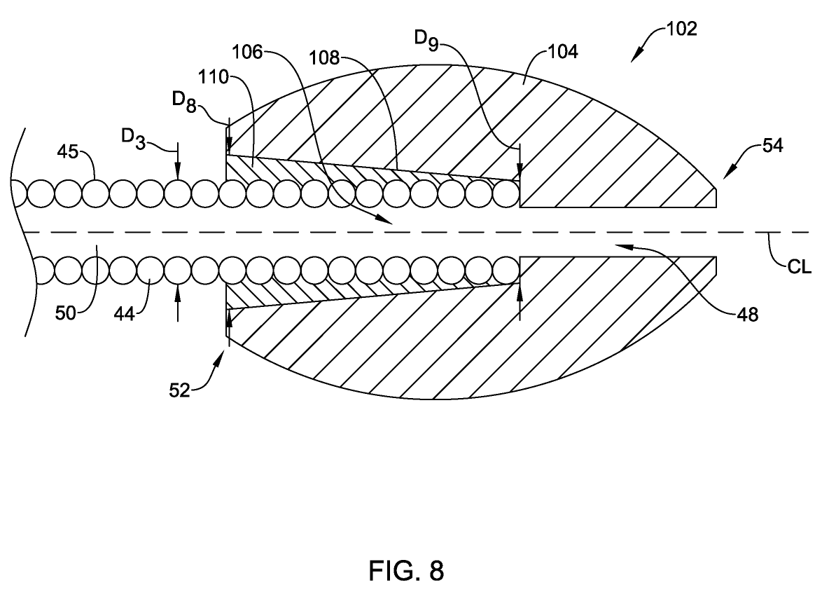

FIG. 8 is a schematic cross-sectional view of an illustrative atherectomy system 102. The illustrative atherectomy system 102 includes an atherectomy burr 104 that is secured to the driveshaft 44. The atherectomy system 102 may be used with the atherectomy system 10 shown in FIGS. 1 and 2, with the atherectomy burr 104 replacing the atherectomy burr 20 and the driveshaft 44 replacing the driveshaft 18. The atherectomy burr 104 defines a cavity 106 that extends distally into the atherectomy burr 104. The guidewire lumen 48 extends distally from the cavity 106. The guidewire lumen 48 may be considered as being coaxially aligned with the lumen 50 that extends through the driveshaft 44. This allows the atherectomy system 102 to be advanced over the guidewire 34 (FIG. 1). The atherectomy burr 104 may be considered as extending from a proximal end region 52 to a distal end region 54. A centerline CL extends through the atherectomy system 102. Because the atherectomy burr 104 is appropriately aligned with the driveshaft 44, the centerline CL represents both a centerline (or axis of rotation) of the driveshaft 44 as well as a centerline (or axis of rotation) of the atherectomy burr 104.

The cavity 106 has a tapering inner diameter, defined by an inner surface 108 of the cavity 106. The cavity 106 has a maximum inner diameter, labeled as diameter D₈, at a position at or near the proximal end region 52 of the atherectomy burr 104 and a minimum inner diameter, labeled as diameter D₉, at a distal terminus of the cavity 106. In some cases, the minimum inner diameter D₉ may be equal to the outer diameter D₃ of the driveshaft 44 and aligns the atherectomy burr 104 with the driveshaft 44 such that a centerline (or axis of rotation) of the atherectomy burr 104 aligns with the centerline (or axis of rotation) of the driveshaft 44, and thus align with the centerline CL of the atherectomy system 102. Equal or substantially equal may be defined as the minimum inner diameter D₉ being within 0.0050 inches (0.13 millimeters) ± or less of the outer diameter D₃. For example, the minimum inner diameter D₉ may be up to 0.0050 inches (0.13 millimeters) smaller or larger than the outer diameter D₃. An adhesive 110 is disposed within the cavity 106, between an inner surface 108 of the cavity 106 and an outer surface 45 of the driveshaft 44.

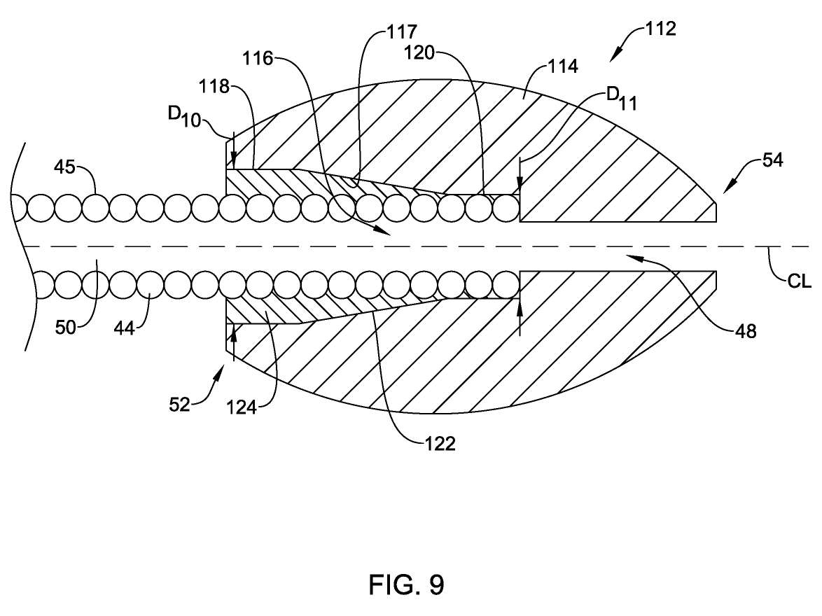

FIG. 9 is a schematic cross-sectional view of an illustrative atherectomy system 112. The illustrative atherectomy system 112 includes an atherectomy burr 114 that is secured to the driveshaft 44. The atherectomy system 112 may be used with the atherectomy system 10 shown in FIGS. 1 and 2, with the atherectomy burr 114 replacing the atherectomy burr 20 and the driveshaft 44 replacing the driveshaft 18. The atherectomy burr 114 defines a cavity 116 that extends distally into the atherectomy burr 114. The guidewire lumen 48 extends distally from the cavity 116. The guidewire lumen 48 may be considered as being coaxially aligned with the lumen 50 that extends through the driveshaft 44. This allows the atherectomy system 112 to be advanced over the guidewire 34 (FIG. 1). The atherectomy burr 114 may be considered as extending from a proximal end region 52 to a distal end region 54. A centerline CL extends through the atherectomy system 112. Because the atherectomy burr 114 is appropriately aligned with the driveshaft 44, the centerline CL represents both a centerline (or axis of rotation) of the driveshaft 44 as well as a centerline (or axis of rotation) of the atherectomy burr 114.

The cavity 116 includes a proximal region 118 having a first inner diameter D₁₀, a distal region 120 having a second inner diameter D₁₁ that is less than the first inner diameter D₁₀, and an intermediate tapered region 122 having a diameter that ranges from the first inner diameter D₁₀ to the second inner diameter D₁₁. In some cases, the second inner diameter D₁₁ is equal to the outer diameter D₃ of the driveshaft 44. Equal or substantially equal may be defined as the second inner diameter D₁₁ being within 0.0050 inches (0.13 millimeters) ± or less of the outer diameter D₃. For example, the second inner diameter D₁₁ may be up to 0.0050 inches (0.13 millimeters) smaller or larger than the outer diameter D₃. A tight fit between an outer surface 45 of the driveshaft 44 and the distal region 120 helps to align the atherectomy burr 114 with the driveshaft 44 such that a centerline (or axis of rotation) of the atherectomy burr 114 aligns with the centerline (or axis of rotation) of the driveshaft 44, and thus align with the centerline CL of the atherectomy system 112. An adhesive 124 is disposed within the cavity 116, between an inner surface 117 of the cavity 116 and an outer surface 45 of the driveshaft 44.

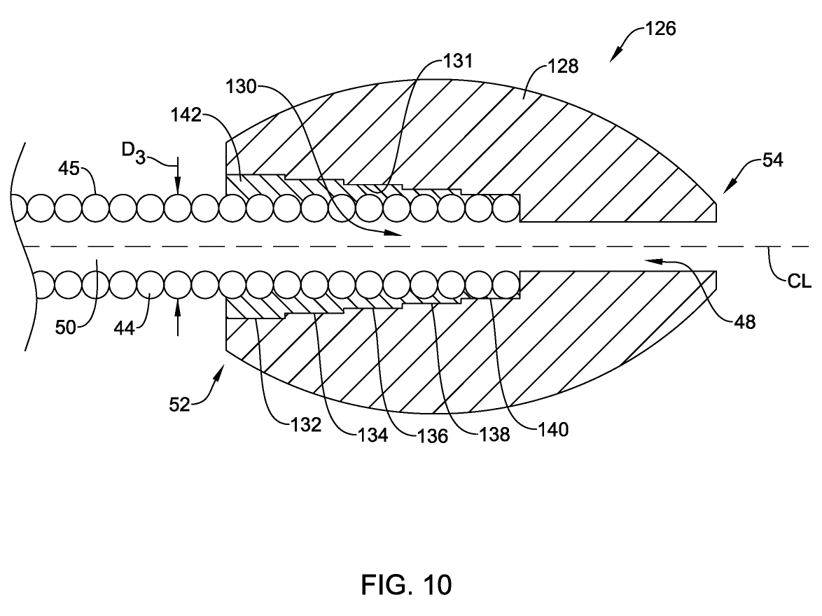

FIG. 10 is a schematic cross-sectional view of an illustrative atherectomy system 126. The illustrative atherectomy system 126 includes an atherectomy burr 128 that is secured to the driveshaft 44. The atherectomy system 126 may be used with the atherectomy system 10 shown in FIGS. 1 and 2, with the atherectomy burr 128 replacing the atherectomy burr 20 and the driveshaft 44 replacing the driveshaft 18. The atherectomy burr 128 defines a cavity 130 that extends distally into the atherectomy burr 128. The guidewire lumen 48 extends distally from the cavity 130. The guidewire lumen 48 may be considered as being coaxially aligned with the lumen 50 that extends through the driveshaft 44. This allows the atherectomy system 126 to be advanced over the guidewire 34 (FIG. 1). The atherectomy burr 128 may be considered as extending from a proximal end region 52 to a distal end region 54. A centerline CL extends through the atherectomy system 126. Because the atherectomy burr 128 is appropriately aligned with the driveshaft 44, the centerline CL represents both a centerline (or axis of rotation) of the driveshaft 44 as well as a centerline (or axis of rotation) of the atherectomy burr 128.

The cavity 130 includes a number of step-wise reductions in diameter. As shown, the cavity 130 includes a first region 132, a second region 134, a third region 136, a fourth region 138 and a fifth region 140. In some cases, there may be fewer than five distinct regions. In some cases, there may be six or more distinct regions. In some cases, some of the regions may include tapered diameters, rather than step-wise changes in diameter. In some cases, the fifth region 140 may have an inner diameter that is equal to the outer diameter D₃ of the driveshaft 44. Equal or substantially equal may be defined as the fifth region 140 having an inner diameter that is within 0.0050 inches (0.13 millimeters) ± or less of the outer diameter D₃ . For example, the diameter of the fifth region 140 may be up to 0.0050 inches (0.13 millimeters) smaller or larger than the outer diameter D₃. A tight fit between an outer surface 45 of the driveshaft 44 and the fifth region 140 helps to align the atherectomy burr 128 with the driveshaft 44 such that a centerline (or axis of rotation) of the atherectomy burr 128 aligns with the centerline (or axis of rotation) of the driveshaft 44, and thus align with the centerline CL of the atherectomy system 126. An adhesive 142 is disposed within the cavity 130, between an inner surface 131 of the cavity 130 and an outer surface 45 of the driveshaft 44.

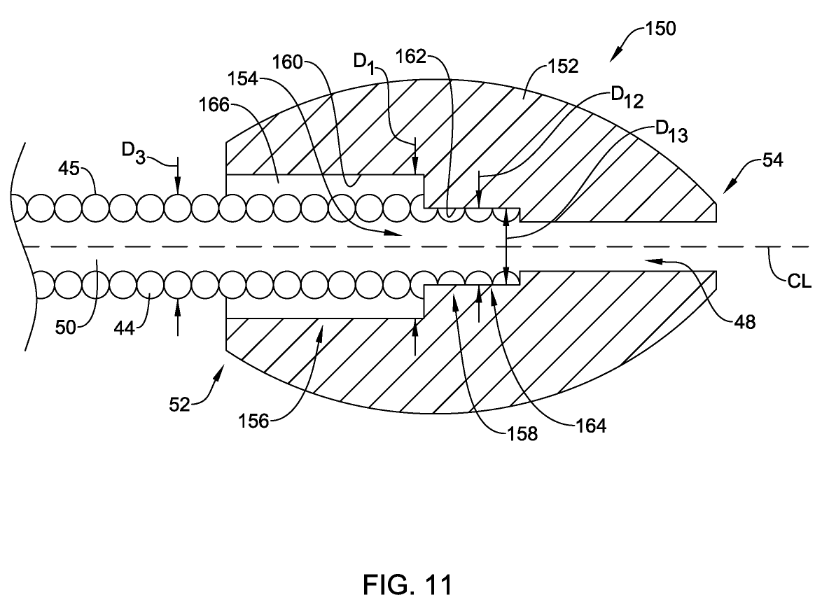

FIG. 11 is a schematic cross-sectional view of an illustrative atherectomy system 150. The illustrative atherectomy system 150 includes an atherectomy burr 152 that is secured to a driveshaft 44. The atherectomy system 150 may be used with the atherectomy system 10 shown in FIGS. 1 and 2, with the atherectomy burr 152 replacing the atherectomy burr 20 and the driveshaft 44 replacing the driveshaft 18. As shown, the driveshaft 44 is a drive coil. The atherectomy burr 152 defines a cavity 154 that extends distally into the atherectomy burr 152. A guidewire lumen 48 extends distally from the cavity 154 and is coaxially aligned with the cavity 46. The guidewire lumen 48 may be considered as being coaxially aligned with a lumen 50 that extends through the driveshaft 44. This allows the atherectomy system 150 to be advanced over the guidewire 34 (FIG. 1). The atherectomy burr 152 may be considered as extending from a proximal end region 52 to a distal end region 54. A centerline CL extends through the atherectomy system 150. Because the atherectomy burr 152 is appropriately aligned with the driveshaft 44, the centerline CL represents both a centerline (or axis of rotation) of the driveshaft 44 as well as a centerline (or axis of rotation) of the atherectomy burr 152.

As shown, the cavity 154 includes a proximal region 156 that has a first inner diameter D₁ and a distal region 158 that has a second inner diameter D₁₂. The proximal region 156 includes an inner surface 160 and the distal region 158 includes an inner surface 162. In some cases, the second inner diameter D₁₂ is less than the first inner diameter D₁. In some cases, the distal region 156 having the second inner diameter D₁₂ may be formed during a process in which the cavity 154, including the proximal region 156 and the distal region 158, are formed by removing material from the atherectomy burr 152. This may occur via drilling, grinding or milling, for example. In some cases, the driveshaft 44 has an outer diameter D₃. In some cases, a portion of the driveshaft 44 may have an outer diameter D₁₃ where a portion of the driveshaft 44 has been milled away. In some cases, the outer diameter D₃ is equal or substantially equal to the second inner diameter D₁₃ . Equal or substantially equal may be defined as the second inner diameter D₁₃ being within 0.0050 inches (0.13 millimeters) ± or less of the outer diameter D₃. For example, the second inner diameter D₁₂ may be up to 0.0050 inches (0.013 centimeters) smaller or larger than the outer diameter D₃. A tight fit between an outer surface 164 of the driveshaft 44 and the distal region 162 helps to align the atherectomy burr 152 with the driveshaft 44 such that a centerline (or axis of rotation) of the atherectomy burr 152 aligns with the centerline (or axis of rotation) of the driveshaft 44, and thus aligns with the centerline CL of the atherectomy system 152. An adhesive 166 is disposed within the cavity 154, between the inner surfaces 160 and 162 of the proximal region 154 and the distal region 158, respectively, of the cavity 154 and the outer surface 45 of the driveshaft 44.

The materials that can be used for the various components of the devices and the various elements thereof disclosed herein may include those commonly associated with medical devices. In some instances, the devices described herein, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility may be imparted into the devices and/or other elements disclosed herein. For example, the devices described herein, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An atherectomy system, comprising:
an atherectomy burr extending from a proximal end region to a distal end region, the atherectomy burr including a burr centerline;
a cavity extending distally into the atherectomy burr from the proximal end region;
a driveshaft extending into the cavity, the driveshaft including a driveshaft centerline; and
an adhesive disposed within the cavity, the adhesive securing the atherectomy burr to the driveshaft;
wherein at least one of the cavity and/or the driveshaft are adapted to align the driveshaft centerline with the burr centerline.

2. The atherectomy system of claim 1, further comprising a guidewire lumen extending distally from and coaxial with the cavity.

3. The atherectomy system of claim 1, further comprising a drive assembly adapted to rotate the driveshaft over a guidewire extending through the guidewire lumen.

4. The atherectomy system of claim 1, wherein the cavity includes:
a proximal region having a first inner diameter; and
a distal region having a second inner diameter that is less than the first inner diameter.

5. The atherectomy system of claim 4, wherein the driveshaft has an outer diameter that is equal to the second inner diameter.

6. The atherectomy system of claim 4, wherein the distal region is created when removing material from the atherectomy burr to form the cavity.

7. The atherectomy system of claim 4, wherein the distal section is formed by removing material from the atherectomy burr to form a constant diameter cavity, followed by disposing an annular ring within the cavity.

8. The atherectomy system of claim 2, wherein;
the cavity has a constant inner diameter;
the atherectomy burr includes an extension extending proximally within the cavity, the extension defining an inner diameter aligned with the guidewire lumen and an outer diameter; and
the driveshaft includes a proximal portion having an outer diameter equal to the constant inner diameter of the cavity, and a distal portion having a reduced inner diameter that matches the reduced inner diameter defined by the extension.

9. The atherectomy system of claim 8, wherein the distal portion of the driveshaft is formed by removing material from an inner surface of the driveshaft.

10. The atherectomy system of claim 1, wherein:
the cavity has a constant inner diameter; and
the driveshaft includes:
a proximal section having a constant outer diameter
a flared distal section having an increasing outer diameter, the flared distal section having a maximum outer diameter that is equal to the constant inner diameter of the cavity.

11. The atherectomy system of claim 1, wherein:
the cavity has a constant inner diameter;
the driveshaft has a constant outer diameter; and
a metal ring is welded to the driveshaft, the metal ring having an outer diameter that is equal to the constant inner diameter of the cavity.

12. The atherectomy system of claim 1, wherein:
the cavity has a tapering inner diameter, with a maximum inner diameter at a proximal end of the cavity and a minimum inner diameter at a distal end of the cavity.

13. The atherectomy system of claim 1, wherein:
the cavity includes:
a proximal region having a first inner diameter;
a distal region having a second inner diameter that is less than the first inner diameter; and
an intermediate tapered region; and
wherein the driveshaft has an outer diameter that is equal to the second inner diameter.

14. The atherectomy system of claim 1, wherein:
the cavity includes a plurality of step-wise reductions in diameter, ranging from a maximum inner diameter at the proximal end of the cavity to a minimum inner diameter at the distal end of the cavity; and
the driveshaft has an outer diameter equal to the minimum inner diameter.

15. The atherectomy system of claim 1, wherein the atherectomy burr is symmetric about the burr centerline.

16. The atherectomy system of claim 1, wherein the atherectomy burr is asymmetric about the burr centerline.

17. An atherectomy system, comprising:
an atherectomy burr;
a cavity extending distally into the atherectomy burr, the cavity having a varying inner diameter including a minimum inner diameter;
a driveshaft extending into the cavity, the driveshaft having an outer diameter equal to the minimum inner diameter; and
an adhesive disposed within the proximal region of the cavity, the adhesive securing the atherectomy burr to the driveshaft.

18. The atherectomy system of claim 17, wherein the driveshaft comprises a drive coil.

19. The atherectomy system of claim 17, wherein the cavity includes a step-wise change in diameter from the proximal region to the distal region.

20. An atherectomy system, comprising:
an atherectomy burr;
a cavity extending distally into the atherectomy burr, the cavity including:
a proximal region having a proximal region inner diameter; and
a distal region having a distal region inner diameter that is less than the proximal region inner diameter;
a driveshaft extending into the cavity, the driveshaft having an outer diameter equal to the distal region inner diameter; and
an adhesive securing the atherectomy burr to the driveshaft.
